(19) United States
(12) Patent Application Publication
(10) Pub. No.: US 2012/0028345 A1
(43) Pub. Date: Feb. 2, 2012

(54) SOLID STATE FERMENTATION (SSF) SYSTEM

(76) Inventors: Darah Ibrahim, Penang (MY); Ibrahim Che Omar, Penang (MY); Chee Keong Lee, Penang (MY); Pei Kheng Pang, Penang (MY)

(21) Appl. No.: 13/201,538

(22) PCT Filed: Feb. 18, 2009
(86) PCT No.: PCT/MY09/00031

§ 371 Date: Sep. 29, 2011

(51) Int. Cl. C12M 1/00

(52) U.S. Cl. 435/289.1

(57) ABSTRACT

The present invention relates to a solid state fermentation system. The system comprises a fermenter module having plural port means for allowing inlet and outlet of substrate to be fermented and control module being operatively inter-engaged with said fermenter module in a manner so as to provide control of fermentation parameters.

### FIELD OF INVENTION

The present invention relates to a solid state fermentation (SSF) system. More particularly, the invention relates to a system for solid-state fermentation.

### BACKGROUND ART

Generally, solid state fermentation is used to cultivate microorganisms for separation of microorganisms themselves or the metabolic product or a microbial altered substrate e.g. in the food-processing industry. The SSF is defined as growth of microorganisms, usually fungi on solid substrates in a defined gas phase, but in absence or near absence of free water phase. Current decade has witnessed an unprecedented spurt in SSF for the development of bioprocesses such as bioremediation and biodegradation of hazardous compounds, biological detoxification of agro-industrial residues, biotransformation of crops and crop-residues for nutritional enrichment, biopulping and production of value-added products such as biologically active secondary metabolites, including antibiotics, alkaloids, plant growth factors, enzymes, organic acids, biosurfactants, aroma compounds etc.

SSF was already used for the production of fermented food, of enzyme products or of edible mushrooms in certain territories of the Orient, Asia and Africa in the Ancient World. However, many institutes and firms recently show their interest in the SSF, because there are certain advantages compared to the submerged fermentation. Such advantages compared to the submerged fermentation are possibility of an effective production of secondary metabolites such as enzymes, aromatic substances and coloring substances as well as pharmaceutically active substances, possibility of production of microorganisms in the form of biological agents in agricultural pesticides or elimination of toxins or other detrimental substances from food stuff or enrichment of proteins or vitamins of this stuff.

However, the application and utilization of agricultural and other domestic wastes through fermentation processes and enzymatic conversion are limited due to their low volume systems. Therefore, a good and reproducible solid state fermentation (SSF) is needed. The SSF fermenters as available at present are relatively limited in the market worldwide. Generally, three types of fermenters are available which are tray fermenters, rotary drum fermenters and fluidized bed fermenters:

Tray fermenters usually using wooden, plastic or metal trays are simple and widely used in traditional SSF. The cultivation is done in stationary trays with no mechanical agitation.

In rotary drum fermenters, the drum rotates intermittently during cultivation to agitate and mix the substrate.

In the fluidized bed fermenter, the solid substrate is fluidized by upward airflow.

### Disadvantages of the Existing SSF Systems

1. There are not many SSF systems on market. Normally they are available as custom-made equipments, meaning that they are only meant for one purpose only.
2. In term of maintenance, the existing SSF systems are rather difficult to maintain, as they have no in situ sterilization systems.
3. Many existing SSF systems are based on shallow tray systems, which make mixing process a difficult if not impossible task.
4. In term of sterility, the existing SSF systems are only sterile at the initial stage and not thereafter.

### Disadvantages of Submerged Fermentation System

| | |
|---|---|
| 1. Microorganisms and substrates | Need to be agitated continuously |
| 2. Water usage | Unlimited use |
| 3. Oxygen supply | Aeration |
| 4. Volume to fermentation mash | Larger |
| 5. Liquid waste produced | Larger |
| 6. Physical energy requirement | High |
| 7. Human energy requirement | Low |
| 8. Capital investment | High |

Most manufactures are particularly interested in producing submerged fermentation systems. Therefore, a need exits to overcome the need for mass production of microbial metabolites that have industrial potential via SSF.

The present inventors have found that a system (hereinafter referred to as “FERMSOSTAT”) for cultivation and fermentation processes can have the ability to control different fermentation parameters and avoid contamination when uniquely designed fermenters are introduced for appropriate parametric control in the fermentation process.

### OBJECTS OF THE INVENTION

Accordingly, one object of the present invention is to overcome the disadvantages/drawbacks of the prior art.

Another object of the present invention is to provide a system having unique design for appropriate control over fermentation parameters and avoid contamination.

Another object of the present invention is to provide a feasible and low cost system for solid state fermentation processes.

Yet another object of the present invention is to provide a low energy consumption and environmental friendly system in the solid state fermentation processes.

Yet another object of the present invention is to provide a system with unique design for a wide range of applications in the solid state fermentation process.

### SUMMARY OF THE INVENTION

According to one aspect, the present invention relates to a system for use in a solid state fermentation process, said system comprising

(a) a fermenter module having plural port means for allowing inlet and outlet of substrate to be fermented; and

(b) a control module being operatively inter-engaged with said fermenter module in a manner so as to provide control of fermentation parameters.

### DETAILED DESCRIPTION OF THE PREFERRED EMBODIMENTS

The present invention relates to a system for use in a solid state fermentation (SSF) process. The system comprises a fermenter module which has plural port means for allowing inlet and outlet of substrate to be fermented. The fermenter module comprises a fermenter vessel which accommodates the substrate to be fermented. The fermenter module is operatively inter-engaged with a control means which provide control of fermentation parameters. The fermenter vessel comprises a viewing glass provided over a predetermined superficial area on the surface of the vessel thereby aiding in realizing the current state of the substrate which is undergoing fermentation process inside the vessel.

Another embodiment of the present invention describes plurality of port means operatively engaged to the fermenter vessel. The port means includes substrate port and sampling port where the substrate port is adapted to communicate substrate to be fermented into the fermenter vessel. The biomass and extraction solution flow out through the sampling port.

Another embodiment of the present invention describes a control means in electro-mechanical inter-engagement with the fermenter vessel. The control means is adapted for controlling temperature, agitation rate, stirring frequency, outlets for sampling, inlets for inoculum and substrate addition.

Another embodiment of the present invention describes the fermenter vessel operatively equipped with speed control motor means. The motor means controls the speed of mixing the substrate during solid state fermentation process in the fermenter vessel.

### EXAMPLE

The performance of the system or “FERMSOSTAT” has been evaluated for the production of cellulase and xylanase enzymes by Aspergillus niger USM A1 1. After using the optimized conditions [500 g of substrate (combination of palm kernel cake and sugarcane bagasse at 1:1 ratio), moisture content of 75%, incubation period for 4 days at 30° C.], about 81 U/g of cellulases and 492 U/g of xylanases were produced, respectively.

The invention has been described in a preferred form only and many variations may be made in the invention which will still be comprised within its spirit. The invention is not limited to the details cited above. The components herein described may be replaced by its technical equivalence and yet the invention can be performed. The structure thus conceived is susceptible of numerous modifications and variations, all the details may furthermore be replaced with elements having technical equivalence. In practice the materials and dimensions may be any according to the requirements, which will still be comprised within its true spirit.

FERMSOSTAT is a portable of SSF unit which can be used for the cultivation and fermentation processes of fungi using solid substrates such as agro-waste materials or domestic wastes. The unit acts as a bioreactor/fermenter for the bioconversion of solid wastes into useful products. The unit is equipped with sets of control system for temperature, agitation speed, mixing/stirring frequency and also outlets for sampling and inlets for inoculum and substrate additions. The FERMSOSTAT is an automatic equipment with one push button system and does not need a technician to operate it.

### ADVANTAGES OF THIS INVENTION

1. This invention is new and is not an improvement of any existing product.
2. This invention has been designed to be applicable for a wide range of SSF processes such as the production of enzymes and other microbial metabolites, for the production of flavoring compounds, biomass, animal feeds, compost, antimicrobial compounds and also production of fermentable sugars. It is also applicable for fermentation processes of coffee and cocoa beans, in order to enhance their flavoring properties.
3. This prototype represents an economical, feasible, low cost system, low energy consumption and environmental friendly in the development of SSF processes.

1. A system for use in a solid state fermentation process, said system comprising
(a) a fermenter module having plural port means for allowing inlet and outlet of substrate to be fermented; and
(b) a control module being operatively inter-engaged with said fermenter module in a manner so as to provide control of fermentation parameters.

2. The system as claimed in claim 1 wherein said fermenter module comprises a fermenter vessel adapted for accommodating and mixing predetermined volume of the substrate.

3. The system as claimed in claim 1, wherein said fermenter vessel further comprises viewing glass provided over a predetermined superficial area on the vessel surface in a manner that substrate inside the vessel is visible from outside.

4. The system as claimed in claim 1 wherein said port means comprises a) substrate port and b) sampling port.

5. The system as claimed in claim 4 wherein said substrate port is adapted to communicate substrate to be fermented into the fermenter vessel.

6. The system as claimed in claim 4 wherein said sampling port is adapted to provide a flow out passage for biomass and extraction solution.

7. The system as claimed in claim 1 wherein said control means are adapted for controlling temperature.

8. The system as claimed in claim 1 wherein said control means are further adapted to control agitation rate.

9. The system as claimed in claim 1 wherein said control means are further adapted to control stirring frequency.

10. The system as claimed in claim 1 wherein said control means are further adapted to control outlets for sampling.

11. The system as claimed in claim 1 wherein said control means are further adapted to control inlets for inoculum and substrate addition.

12. The system as claimed in claim 1, further comprising a speed control motor.

13. The system as claimed in claim 1, further comprising a spraying nozzle.

14. A The system as claimed in claim 1, further comprising an impeller.

15. The system as claimed in claim 1, further comprising a mixing control panel.

16. The system as claimed claim 1, further comprising a flowmeter.

17. A The system as claimed in claim 1, adapted for production of microbial metabolites, flavoring compounds, biomass, animal feeds, compost, antimicrobial compounds and/or fermentable sugars.
